(19) United States
(12) Patent Application Publication
(10) Pub. No.: US 2023/0277292 A1
(43) Pub. Date: Sep. 7, 2023

(54) ARTIFICIAL BLOOD VESSEL

(71) Applicant: HI-LEX CORPORATION, Takarazuka-shi, Hyogo (JP)

(72) Inventor: Shinsaku Koarashi, Hyogo (JP)

(21) Appl. No.: 17/928,501

(22) PCT Filed: Jun. 29, 2021
(86) PCT No.: PCT/JP2021/024432

§ 371 Date: Nov. 29, 2022

(30) Foreign Application Priority Data
Jun. 29, 2020 (JP) 2020-111686
Jun. 21, 2021 (JP) 2021-102650

(51) Int. Cl. A61F 2/06; D03D 1/00

(52) U.S. Cl. CPC A61F 2/06; D03D 1/00; D10B 2509/00

(57) ABSTRACT

An artificial blood vessel is used, which alternatingly has, in an extending direction D2 of a weft yarn 2, a first region in which a warp yarn 1 and the weft yarn 2 are woven in a plain weave, a second region having a first portion on the second region side R21 in which the warp yarn 1 crosses over a plurality of weft yarns 2 and a second portion on the second region side R22 in which the warp yarn 1 extends so as to cross over one weft yarn 2, and a third region having a first portion on the third region side R31 in which the warp yarn 1 crosses over a plurality of warp yarns 2 and a second portion on the third region side R32 in which the warp yarn 1 extends so as to cross over one weft yarn 2.

### TECHNICAL FIELD

The present invention relates to an artificial blood vessel.

### BACKGROUND ART

The artificial blood vessel is used, for example, for replacing a pathological living blood vessel. The artificial blood vessel is required to have biocompatibility and flexibility, as well as little blood leakage from the artificial blood vessel, that is, a high blood leakage resistance. Most of general polyester artificial blood vessels (fabric artificial blood vessels) are woven with plain weave fibers (see, for example, Patent Document 1), improving blood leakage resistance with addition of a coating, a sealing layer, or the like.

### PRIOR ART DOCUMENT

### Patent Document

Patent Document 1: JP 2012-139498 A

### SUMMARY OF THE INVENTION

### Problem to Be Solved by the Invention

An artificial blood vessel of a plain weave structure is stable as a structure, but there is a limitation to how finely a weft yarn can be packed, and, in particular, porosities at four corners of an intersection part between a warp yarn and a weft yarn become large. Thus, a high blood leakage resistance required for the artificial blood vessel cannot be maintained only with the structure of the artificial blood vessel, and a coating is required for improving the blood leakage resistance.

Therefore, it is an object of the present invention to provide an artificial blood vessel with a weaving structure partially having a plain weave region, the artificial blood vessel having improved blood leakage resistance with a three-dimensional structure of a warp yarn.

### Means to Solve the Problem

The present invention is an artificial blood vessel having a warp yarn and a weft yarn, the artificial blood vessel alternatingly having, in an extending direction of the weft yarn, a first region in which the warp yarn and the weft yarn are woven in a plain weave, a second region having a first portion on the second region side in which the warp yarn crosses over a plurality of weft yarns on one surface of the artificial blood vessel and a second portion on the second region side in which the warp yarn extends so as to cross over one weft yarn on one surface of the artificial blood vessel, and a third region having a first portion on the third region side in which the warp yarn crosses over a plurality of weft yarns on one surface of the artificial blood vessel and a second portion on the third region side in which the warp yarn extends so as to cross over one weft yarn and extends on one surface of the artificial blood vessel, wherein the first portion on the second region side is adjacent to the second portion on the third region side in the extending direction of the weft yarn, and the second portion on the second region side is adjacent to the first portion on the third region side in the extending direction of the weft yarn, and wherein the warp yarn is composed of a multifilament yarn.

### Effects of the Invention

According to the artificial blood vessel of the present invention, in the artificial blood vessel with a weaving structure partially having a plain weave region, blood leakage resistance can be improved with a three-dimensional structure of a warp yarn.

### BRIEF DESCRIPTION OF THE DRAWINGS

FIG. 1 is a weaving structure diagram of an artificial blood vessel according to one embodiment of the present invention.

FIG. 2 is a SEM photograph of an outer surface of the artificial blood vessel having a weaving structure shown in FIG. 1.

FIG. 3 is a weaving structure diagram of the artificial blood vessel according to another embodiment of the present invention.

FIG. 4 is a weaving structure diagram of the artificial blood vessel according to another embodiment of the present invention.

FIG. 5 is a weaving structure diagram of the artificial blood vessel according to another embodiment of the present invention.

### EMBODIMENT FOR CARRYING OUT THE INVENTION

Hereinafter, the artificial blood vessel according to one embodiment of the present invention will be described with reference to the drawings. Besides, embodiments shown below are merely examples, and the artificial blood vessel of the present invention is not limited to the following embodiments.

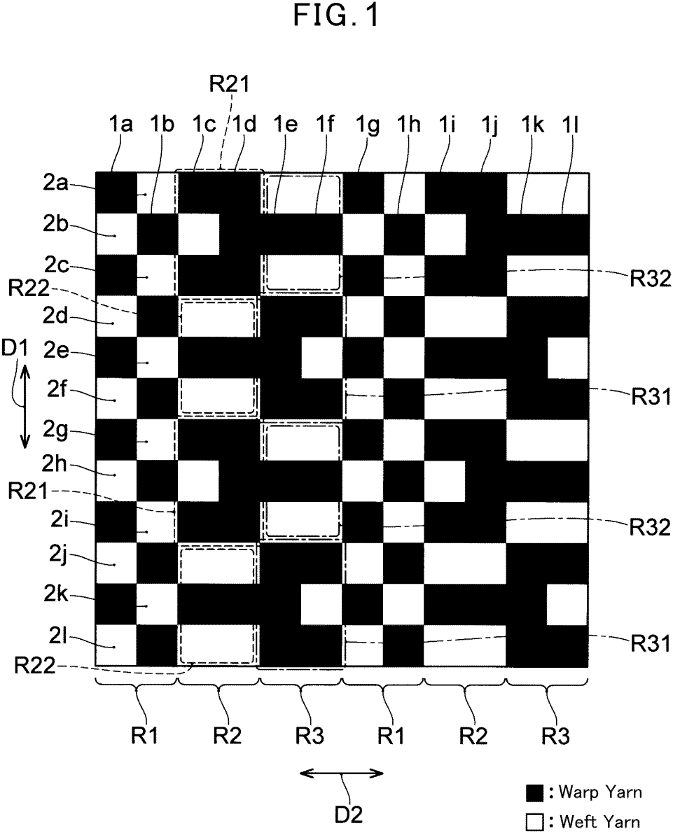

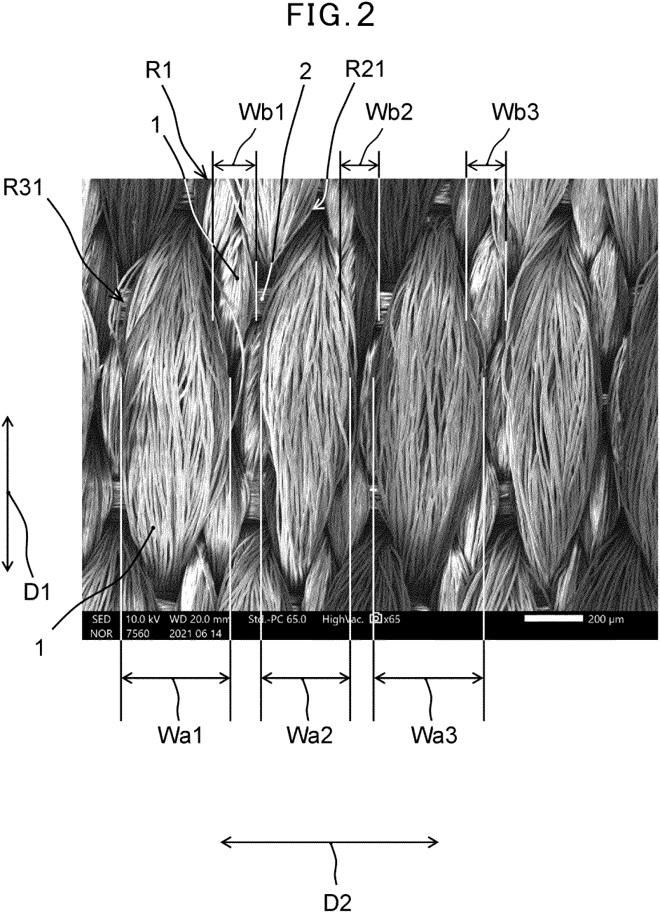

FIG. 1 is a weaving structure diagram of the artificial blood vessel according to one embodiment of the present invention. In FIG. 1, a part (region of 12 warp yarns and 12 weft yarns) of the outer surface of the artificial blood vessel are shown. It should be noted that in FIG. 1, shown in black are parts where the warp yarn is exposed on the outer surface of the artificial blood vessel, and shown in white are parts where the weft yarn is exposed on the outer surface of the artificial blood vessel. FIG. 2 is a SEM photograph of the outer surface of the artificial blood vessel having the weaving structure shown in FIG. 1.

The artificial blood vessel is used, such as, for example, for replacing a pathological living blood vessel and bypassing the living blood vessel. In the present embodiment, the artificial blood vessel is formed of a weaving structure of fibers. As shown in FIG. 1, the artificial blood vessel of the embodiment has warp yarns 1a to 1l (hereinafter collectively referred to as a warp yarn 1) and weft yarns 2a to 2l (hereinafter collectively referred to as a weft yarn 2) and has a weaving structure in which the warp yarn 1 and the weft yarn 2 are interlaced. It should be noted that in FIG. 1, the warp yarn 1 extends in a vertical direction, and an extending direction of the warp yarn 1 is referred to as D1. Moreover, in FIG. 1, the weft yarn 2 extends in a horizontal direction, and the extending direction of the weft yarn 2 is referred to as D2. In the artificial blood vessel, the warp yarn 1 is woven along an axial direction of the artificial blood vessel, and the weft yarn 2 is woven along a circumferential direction of the artificial blood vessel, which are not shown in the drawings. It should be noted that a loom for manufacturing the artificial blood vessel is not particularly limited. A size of the artificial blood vessel of the embodiment is not particularly limited. For example, the artificial blood vessel may be an artificial blood vessel with a large diameter having an inner diameter of 10 mm or more (for a thoracoabdominal aorta), an artificial blood vessel with a medium diameter having an inner diameter of 6 mm or more and less than 10 mm, such as 6 mm and 8 mm (for arteries in lower limb, neck, and axillary regions), or an artificial blood vessel with a small diameter having an inner diameter of less than 6 mm. An axial length of the artificial blood vessel is not particularly limited and can be appropriately changed depending on its intended use.

In the present embodiment, the artificial blood vessel has a first region R1 in which the warp yarn 1 and the weft yarn 2 are woven in a plain weave, as shown in FIG. 1. Moreover, the artificial blood vessel has a second region R2 having a first portion on the second region side R21 in which the warp yarn 1 crosses over a plurality of weft yarns 2 on one surface of the artificial blood vessel (the outer surface of the artificial blood vessel in the present embodiment), and a second portion on the second region side R22 in which the warp yarn extends so as to cross over one weft yarn on one surface of the artificial blood vessel. Furthermore, the artificial blood vessel has a third region R3 having a first portion on the third region side R31 in which the warp yarn 1 crosses over a plurality of weft yarns 2 on one surface of the artificial blood vessel (the outer surface of the artificial blood vessel in the present embodiment) and a second portion on the third region side R32 in which the warp yarn 1 extends so as to cross over one weft yarn 2 on one surface of the artificial blood vessel. The first region R1, the second region R2, and the third region R3 are alternately formed in the extending direction D2 of the weft yarn 2, as shown in FIG. 1. That is, the first region R1, the second region R2, and the third region R3 are repeatedly arranged in this order in the extending direction D2 of the weft yarn 2. The first portion on the second region side R21 is adjacent to the second portion on the third region side R32 in the extending direction D2 of the weft yarn 2, and the second portion on the second region side R22 is adjacent to the first portion on the third region side R31 in the extending direction D2 of the weft yarn 2. In addition, the warp yarn 1 is composed of a multifilament yarn as shown in FIG. 2. With the above-described configuration, in the artificial blood vessel of the embodiment, the warp yarn 1 composed of a multifilament yarn, which extends long without being restrained in the first portion on the second region side R21 or the first portion on the third region side R31, spreads toward the first region R1 woven in a plain weave (and in a direction perpendicular to one surface of the artificial blood vessel, namely, toward front direction of a paper surface in FIGS. 1 and 2) (see FIG. 2), as will be described later. With this three-dimensional structure of the warp yarn 1, when blood seeps out from a gap between fibers generated in the first region R1 woven in a plain weave, the blood is suppressed from leaking out and retained in the three-dimensional structure. By coagulating the blood with being retained, blood leakage resistance can be improved. A configuration and a weaving structure of each part of the artificial blood vessel will be described below.

The warp yarn 1 is a fiber extending in one direction, among fibers constituting the artificial blood vessel. In the present embodiment, the warp yarn 1 is a fiber extending in an axial direction of the artificial blood vessel. The warp yarn 1 is made of a material applicable to a fabric artificial blood vessel composed of a weaving structure of fibers. The material of the warp yarn 1 is not particularly limited as long as it is a material applicable to the fabric artificial blood vessel. For example, the material of the warp yarn 1 can be polyester, polytetrafluoroethylene, polyamide, or the like. Moreover, a composite material composed of two or more kinds of applicable materials having different properties such as a melting point and a degree of shrinkage may be used. For example, the composite material may be a synthetic fiber in which polyethylene terephthalate (PET) and polytrimethylene terephthalate (PTT), etc. are combined at a spinning stage to form one long filament having a spiral crimp. For example, when the composite material composed of two kinds of materials having different melting point and degree of shrinkage, which has a spiral crimp, is used as a material of the warp yarn 1, a three-dimensional structure composed of a warp yarn 1 which will be described later is easy to spread in the extending direction D2 of the weft yarn 2, further enhancing a performance of retaining blood, which can improve the blood leakage resistance.

Each of the warp yarns 1 is composed of a multifilament yarn in the present embodiment (see FIG. 2). A fineness of the warp yarn 1 is not particularly limited as long as the filament of the warp yarn 1 can spread toward the first region R1 to close the gap between the fibers. The fineness of the warp yarn 1 can be, for example, 0.25 to 2.50 dtex, preferably 0.50 to 2.00 dtex, for a single yarn fineness of the warp yarn 1, and can be 2 to 2500 dtex, preferably 6 to 1600 dtex, more preferably 10 to 540 dtex, further preferably 30 to 200 dtex, for a total fineness of the warp yarns 1. When the single yarn fineness of the warp yarn 1 and the total fineness of the warp yarns 1 are within the above-described ranges, the warp yarns 1 of the second region R2 and the third region R3 can be satisfactorily spread toward the first region R1. Therefore, when blood seeps out from a gap in the first region R1 with the warp yarns 1 of the second region R2 and the third region R3, the blood is suppressed from leaking out, retained by the three-dimensional structure of the warp yarn 1, and coagulates in the retained state, which can improve the blood leakage resistance. It should be noted that the “single yarn fineness” is a fineness per single filament constituting the warp yarn 1, and the “total fineness” is a product of the single yarn fineness and the number of filaments constituting the warp yarn 1. The number of filament yarns (hereinafter referred to as the number of filaments) constituting one warp yarn is not particularly limited. For example, as will be described later, when the total number of filaments of the warp yarns 1 is 1.5 times or more the number of filaments per single weft yarn 2 and the number of warp yarns 1 crossing over a plurality of weft yarns 2 is one in the second region R2, the number of filaments per single warp yarn 1 can be 8 to 1000, preferably 12 to 800, more preferably 20 to 270, further preferably 60 to 100. As will be described later, when the number of filaments per single warp yarn 1 is 0.8 to 1.2 times the number of filaments per single weft yarn 2 and the number of warp yarns 1 crossing over a plurality of weft yarns 2 is two or more in the second region R2, the number of filaments per single warp yarn 1 can be 4 to 500, preferably 6 to 400, more preferably 10 to 135, further preferably 30 to 50.

The weft yarn 2 is a fiber extending in a direction intersecting with the warp yarn 1, among fibers constituting the artificial blood vessel. In the present embodiment, the weft yarn 2 is a fiber extending in a circumferential direction of the artificial blood vessel. The weft yarn 2 is made of a material applicable to a fabric artificial blood vessel composed of a weaving structure of fibers. The material of the weft yarn 2 is not particularly limited as long as it is a material applicable to the fabric artificial blood vessel. For example, the material of the weft yarn 2 can be polyester, polytetrafluoroethylene, polyamide, or the like.

Each of the weft yarns 2 may be a monofilament yarn or a multifilament yarn, but in the present embodiment, the weft yarn 2 is composed of a multifilament yarn as shown in FIG. 2. A fineness of the weft yarn 2 is not particularly limited, but for example, when the weft yarn 2 is a monofilament yarn, a single yarn fineness of the weft yarn 2 can be 15 to 100 dtex, preferably 20 to 75 dtex. Moreover, when each of the weft yarns 2 is composed of a multifilament yarn, for example, the single yarn fineness of the weft yarn 2 can be 0.25 to 2.50 dtex, preferably 0.50 to 2.00 dtex, and a total fineness of the weft yarns 2 can be 1 to 1250 dtex, preferably 3 to 800 dtex, more preferably 5 to 270 dtex, further preferably 15 to 100 dtex. It should be noted that the “single yarn fineness” is a fineness per single filament (monofilament or multifilament) constituting the weft yarn 2, and the “total fineness” is a product of a single yarn fineness and the number of filaments constituting the weft yarn 2. When the weft yarn 2 is composed of a multifilament yarn, the number of filament yarns constituting one weft yarn can be 4 to 500, preferably 6 to 400, more preferably 10 to 135, further preferably 30 to 50.

The first region R1 is a section where the warp yarn 1 and the weft yarn 2 are plain-woven. In FIG. 1, the first region R1 is a region where the warp yarns 1a, 1b, 1g, 1h and the weft yarns 2 (weft yarns 2a to 2l) intersect. The first region R1 improves a strength of the artificial blood vessel, particularly a tensile strength (in the axial direction of the artificial blood vessel). The first region R1 extends along the extending direction D1 of the warp yarn 1 and extends in the axial direction of the artificial blood vessel. Moreover, a plurality of first regions R1 are arranged apart from each other at a predetermined interval in the extending direction D2 of the weft yarn 2. The second region R2 and the third region R3 are arranged between one first region R1 and the other first region R1 in the extending direction D2 of the weft yarn 2.

In the present embodiment, as shown in FIG. 1, two warp yarns 1a and 1b (1g, 1h) and a plurality of weft yarns 2a to 2l (and weft yarns not shown) are plain-woven in the first region R1. The number of warp yarns 1 provided in one first region R1 can be 2 to 4, preferably 2 to 3, more preferably 2. It should be noted that, in the present specification, the term “the number of warp yarns” refers to, with the warp yarn 1 composed of a plurality of filament yarns to be bundled being as one, the number of warp yarns 1 in which the filament yarns are bundled, not the number of filaments constituting multifilament yarns. When the number of warp yarns 1 is within the above-mentioned ranges, an uncovered area of the first region R1, which is not covered by the warp yarns 1 of the first portion on the second region side R21 and the warp yarns 1 of the first portion on the third region side R31, can be reduced. Therefore, the first region R1 in plain weave becomes easily covered three-dimensionally with the warp yarn 1 of the first portion on the second region side R21 and the warp yarn 1 of the first portion on the third region side R31. When blood seeps out from the first region R1, the blood is retained by the three-dimensional structure of the warp yarns 1 of the first portion on the second region side R21 and the warp yarns 1 of the first portion on the third region side R31 and coagulates in the retained state. Therefore, an amount of blood leakage from the artificial blood vessel can be reduced. Moreover, in the artificial blood vessel, a ratio of the number of warp yarns 1 in the first region R1 to the total number of warp yarns 1 arranged in the extending direction D2 of the weft yarn 2 in the first region R1 to the third region R3 (the number of warp yarns in the first region R1/the total number of warp yarns) is not particularly limited, but can be, for example, 0.2 to 0.4 (⅓ in the present embodiment). When the number of warp yarns 1 in the first region R1 and the ratio of the number of warp yarns 1 are within the above-described ranges, the amount of blood leakage from the artificial blood vessel can be reduced while increasing the strength of the artificial blood vessel.

The second region R2 has a first portion on the second region side R21 in which the warp yarn 1 crosses over a plurality of weft yarns 2 and a second portion on the second region side R22 in which the warp yarn 1 extends so as to cross over one weft yarn 2. As shown in FIG. 1, the first portion on the second region side R21 and the second portion on the second region side R22 are alternately provided in the extending direction D1 of the warp yarn 1. Since the second region R2 has the first portion on the second region side R21 and the second portion on the second region side R22, the artificial blood vessel can be made more flexible as compared with the artificial blood vessels, all region of which has a plain weave structure. The number of warp yarns 1 provided in the second region R2 can be, for example, 1 to 4, preferably 2 to 3, more preferably 2.

The first portion on the second region side R21 is a portion woven so that the warp yarn 1 has a portion crossing over a plurality of weft yarns 2. In the present embodiment, the warp yarns 1d, 1j, etc. cross over the plurality of weft yarns 2. In the first portion on the second region side R21, the warp yarn 1 crosses over the plurality of weft yarns 2, so that the artificial blood vessel becomes more flexible in that portion than in the plain weave structure. Moreover, the warp yarn 1 of the first portion on the second region side R21 is composed of a multifilament yarn, and both ends of the first portion on the second region side R21 in the extending direction D1 of the warp yarn 1 become in a state of being bound by the weft yarns 2 of the second portion on the second region side R22. Therefore, as shown in FIG. 2, the multifilament yarn bound at both ends thereof forms a three-dimensional structure that spreads in the extending direction D2 of the weft yarn 2 (besides, this three-dimensional structure also spreads in a front direction of a paper surface in FIG. 2). Thus, the first region R1 with the plain weave structure adjacent to the first portion on the second region side R21 in the extending direction D2 of the weft yarn 2 is partially covered with the spread multifilament yarn of the first portion on the second region side R21. With this three-dimensional structure of the warp yarn 1, when blood seeps out from a gap between fibers generated in the plain-woven first region R1, the seeping blood is retained in a gap between filaments of the three-dimensional structure composed of the multifilament. As a result, the blood coagulates in the retained state, so that the blood leakage resistance can be improved. Moreover, in the present embodiment, the second portion on the third region side R32 adjacent to the first portion on the second region side R21 in the extending direction D2 of the weft yarn 2 is also partially covered with the spread multifilament yarn of the first portion on the second region side R21. As a result, a gap generated in the second portion on the third region side R32 is also covered with the multifilament yarn of the first portion on the second region side R21, so that the blood in the artificial blood vessel becomes less likely to leak out to the outside.

In the first portion on the second region side R21 (from a portion where the warp yarn 1 exits from the other surface of the artificial blood vessel to one surface of the artificial blood vessel (the surface shown in FIG. 1), to a portion where the warp yarn 1 enters into the other surface), the number of weft yarns 2 which the warp yarn 1 crosses over is not particularly limited, but can be, for example, 2 to 5, preferably 3 to 4, more preferably 3 (the state shown in FIG. 1). When the number of warp yarns 2 which the warp yarn 1 crosses over is within the above-described ranges, in the first portion on the second region side R21, the multifilament yarn of the warp yarns 1 is easy to be spread in the extending direction D2 of the weft yarn 2, and the strength of the artificial blood vessel can be maintained at a predetermined level.

In the first portion on the second region side R21, the number of warp yarns 1 constituting the first portion on the second region side R21 is not particularly limited as long as the warp yarn 1 has a portion crossing over a plurality of weft yarns 2. In the present embodiment, the first portion on the second region side R21 has a plurality of (two) warp yarns 1c, 1d (or warp yarns 1i, 1j). It should be noted that the second region R2 may have at least one warp yarn 1 extending so as to cross over (only) one weft yarn 2 and at least one warp yarn 1 crossing over a plurality of weft yarns 2. In the present embodiment, the second region R2, like the first region R1 having a plain weave structure, comprises a warp yarn 1c (warp yarn 1i) crossing over only one weft yarn 2 and then extending from one surface of the artificial blood vessel to the other surface thereof and a warp yarn 1d (warp yarn 1j) crossing over a plurality of weft yarns 2 and then extending from one surface of the artificial blood vessel to the other surface thereof.

The second portion on the second region side R22 is a portion woven so that the warp yarn 1 crosses over only one weft yarn 2 (the warp yarn 1 does not cross over a plurality of weft yarns 2 from a portion where it exits from the other surface of the artificial blood vessel to one surface of the artificial blood vessel (the surface shown in FIG. 1) to a portion where it enters into the other surface). The second portion on the second region side R22 is set to have the same degree of length as the length of the first portion on the second region side R21 in the extending direction D1 of the warp yarn 1. That is, the number of weft yarns 2 in the first portion on the second region side R21 (three weft yarns 2 in FIG. 1) is equal to the number of weft yarns 2 in the second portion on the second region side R22 (three weft yarns 2 in FIG. 1).

The third region R3 has a first portion on the third region side R31 in which the warp yarn 1 crosses over a plurality of weft yarns 2 and a second portion on the third region side R32 in which the warp yarn 1 extends so as to cross over one weft yarn 2. As shown in FIG. 1, the first portion on the third region side R31 and the second portion on the third region side R32 are alternately provided in the extending direction D1 of the warp yarn 1. Since the third region R3 has the first portion on the third region side R31 and the second portion on the third region side R32, the artificial blood vessel can be made more flexible as compared with the artificial blood vessels, all region of which has a plain weave structure. The number of warp yarns 1 provided in the third region R3 can be, for example, 1 to 4, preferably 2 to 3, more preferably 2.

The first portion on the third region side R31 is a portion woven so that the warp yarn 1 has a portion crossing over a plurality of weft yarns 2. In the present embodiment, the warp yarns 1e, 1k, etc. cross over the plurality of weft yarns 2. In the first portion on the third region side R31, the warp yarn 1 crosses over the plurality of weft yarns 2, so that the artificial blood vessel becomes more flexible in that portion than in the plain weave structure. Moreover, the warp yarn 1 of the first portion on the third region side R31 is composed of a multifilament yarn, and both ends of the first portion on the third region side R31 in the extending direction D1 of the warp yarn 1 become in a state of being bound by the weft yarns 2 of the second portion on the third region side R32. Therefore, as shown in FIG. 2, the multifilament yarn bound at both ends thereof forms a three-dimensional structure that spreads in the extending direction D2 of the weft yarn 2. Besides, this three-dimensional structure also spreads in a front direction of a paper surface in FIG. 2. Thus, the first region R1 with the plain weave structure adjacent to the first portion on the third region side R31 in the extending direction D2 of the weft yarn 2 is partially covered with the spread multifilament yarn of the first portion on the third region side R31. With this three-dimensional structure of the warp yarn 1, when blood seeps out from a gap between fibers generated in the plain-woven first region R1, the seeping blood is retained in a gap between filaments of the three-dimensional structure composed of the multifilament. As a result, the blood coagulates in the retained state, so that the blood leakage resistance can be improved. Moreover, in the present embodiment, the second portion on the second region side R22 adjacent to the first portion on the third region side R31 in the extending direction D2 of the weft yarn 2 is also partially covered with the spread multifilament yarn of the first portion on the third region side R31. As a result, a gap generated in the second portion on the second region side R22 is also covered with the multifilament yarn of the first portion on the third region side R31, so that the blood in the artificial blood vessel becomes less likely to leak out to the outside.

In the first portion on the third region side R31 (from a portion where the warp yarn 1 exits from the other surface of the artificial blood vessel to one surface of the artificial blood vessel (the surface shown in FIG. 1), to a portion where the warp yarn 1 enters into the other surface), the number of weft yarns 2 which the warp yarn 1 crosses over is not particularly limited, but can be, for example, 2 to 5, preferably 3 to 4, more preferably 3 (the state shown in FIG. 1). When the number of warp yarns 2 which the warp yarn 1 crosses over is within the above-described ranges, in the first portion on the third region side R31, the multifilament yarn of the warp yarns 1 is easy to be spread in the extending direction D2 of the weft yarn 2, and the strength of the artificial blood vessel can be maintained at a predetermined level.

In the first portion on the third region side R31, the number of warp yarns 1 constituting the first portion on the third region side R31 is not particularly limited as long as the warp yarn 1 has a portion crossing over a plurality of weft yarns 2. In the present embodiment, the first portion on the third region side R31 has a plurality of (two) warp yarns 1e, 1f (or warp yarns 1k, 1l). It should be noted that the third region R3 may have at least one warp yarn 1 extending so as to cross over (only) one weft yarn 2 and at least one warp yarn 1 crossing over a plurality of weft yarns 2. In the present embodiment, the third region R3, like the first region R1 having a plain weave structure, comprises a warp yarn 1f (warp yarn 1l) crossing over only one weft yarn 2 and then extending from one surface of the artificial blood vessel to the other surface thereof and a warp yarn 1e (warp yarn 1k) crossing over a plurality of weft yarns 2 and then extending from one surface of the artificial blood vessel to the other surface thereof.

The second portion on the third region side R32 is a portion woven so that the warp yarn 1 crosses over only one weft yarn 2 (the warp yarn 1 does not cross over a plurality of weft yarns 2 from a portion where it exits from the other surface of the artificial blood vessel to one surface of the artificial blood vessel (the surface shown in FIG. 1) to a portion where it enters into the other surface). The second portion on the third region side R32 is set to have the same degree of length as the length of the first portion on the third region side R31 in the extending direction D1 of the warp yarn 1. That is, the number of weft yarns 2 in the first portion on the third region side R31 (three weft yarns 2 in FIG. 1) is the same as the number of weft yarns 2 in the second portion on the third region side R32 (three weft yarns 2 in FIG. 1).

As mentioned above, the artificial blood vessel has a first region R1 in which the warp yarn 1 and the weft yarn 2 are woven in a plain weave, a second region R2 having a first portion on the second region side R21 and a second portion on the second region side R22, and a third region R3 having a first portion on the third region side R31 and a second portion on the third region side R32, alternately in the extending direction D2 of the weft yarn 2. The first portion on the second region side R21 is adjacent to the second portion on the third region side R32 in the extending direction D2 of the weft yarn 2. The second portion on the second region side R22 is adjacent to the first portion on the third region side R31 in the extending direction D2 of the weft yarn 2. The warp yarn 1 is composed of a multifilament yarn. As a result, the multifilament yarn of the warp yarn 1 in the first portion on the second region side R21 spreads in the extending direction D2 of the weft yarn 2 and partially covers the first region R1 adjacent to the first portion on the second region side R21 to fill gaps (porosities) formed at four corners of an intersection part between the warp yarn 1 and the weft yarn 2 formed in the first region R1. Furthermore, the multifilament yarn of the warp yarn 1 in the first portion on the third region side R31 spreads in the extending direction D2 of the weft yarn 2 and partially covers the first region R1 adjacent to the first portion on the third region side R31 to cover the gaps (porosities) formed at the four corners of the intersection part between the warp yarn 1 and the weft yarn 2 formed in the first region R1 with multifilaments in the first portion on the second region side R21 and the first portion on the third region side R31. Therefore, when blood seeps out from a gap between fibers generated in the first region R1 woven in a plain weave, with the three-dimensional structure of the warp yarn 1, the seeping blood is retained in a gap between filaments having a three-dimensional structure composed of multifilaments, allowing for the blood to coagulate without flowing out. Thereby, the blood leakage resistance can be improved. In addition, in the present embodiment, the second portion on the third region side R32 adjacent to the first portion on the second region side R21 is partially covered with the multifilament yarn of the warp yarn 1 in the first portion on the second region side R21 to cover gaps (porosities) formed at an intersection part of the warp yarn 1 and the weft yarn 2 formed in the second portion on the third region side R32. Furthermore, the second portion on the second region side R22 adjacent to the first portion on the third region side R31 is partially covered with the multifilament yarn of the warp yarn 1 in the first portion on the third region side R31 to cover gaps (porosities) formed at an intersection part of the warp yarn 1 and the weft yarn 2 formed in the second portion on the second region side R22. Therefore, the blood in the artificial blood vessel becomes less likely to leak out to the outside through a gap between the second portion on the third region side R32 and the second portion on the second region side R22, improving the blood leakage resistance of the artificial blood vessel.

Furthermore, in the present embodiment, the first region R1 having a plain weave structure, and the second region R2 and the third region R3 each having a weave structure different from the plain weave structure are alternately formed in the extending direction D2 of the weft yarn 2. Therefore, a predetermined flexibility required for the artificial blood vessel can be obtained with the second region R2 and the third region R3, while securing a predetermined strength of the artificial blood vessel with the first region R1 provided at a predetermined interval in the extending direction D2 of the weft yarn 2. Therefore, according to the artificial blood vessel of the embodiment, in addition to improvement of the blood leakage resistance, both strength and flexibility required for the artificial blood vessel can be achieved.

Moreover, in the present embodiment, as shown in FIG. 1, the first portion on the second region side R21 and the first portion on the third region side R31 are configured to extend continuously in the extending direction D1 of the warp yarn 1 in a zigzag shape. In this case, the warp yarn 1 of the first portion on the second region side R21 and the warp yarn 1 of the first portion on the third region side R31, which are spread in the extending direction D2 of the weft yarn 2, do not interfere with each other, and the spread of the warp yarn 1 is not interrupted in the direction D1 of the warp yarn 1, so that absorbability of blood with the three-dimensional structure can be further enhanced.

It is preferable that an average width of the maximum spread of the warp yarn 1 of the first portion on the second region side R21 and the first portion on the third region side R31 in the extending direction D2 of the weft yarn 2 (see Wa1 to Wa3 in FIG. 2) is larger than an average width of the maximum spread of the warp yarn 1 in the first region R1 in the extending direction D2 of the weft yarn 2 (see Wb1 to Wb3). In this case, gaps between the first region R1, the second portion on the second region side R22, and the second portion on the third region side R32 are covered in a large region with the warp yarns 1 of the first portion on the second region side R21 and the first portion on the third region side R31. Therefore, blood seeping out from the gaps between the first region R1, the second portion on the second region side R22, and the second portion on the third region side R32 is easy to be further retained, and the blood leakage resistance can be further improved. It should be noted that the average width of the maximum spread of the warp yarn 1 of the first portion on the second region side R21 and the first portion on the third region side R31 in the extending direction D2 of the weft yarn 2 is not particularly limited, but can be, for example, 2.0 to 4.0 times the average width of the maximum spread of the warp yarn 1 in the region R1 in the extending direction D2 of the weft yarn 2.

It should be noted that the “average width of the maximum spread of the warp yarn 1 of the first portion on the second region side R21 and the first portion on the third region side R31 in the extending direction D2 of the weft yarn 2″ may be obtained by, for example, measuring a predetermined number m of (for example, 10 or more) widths of portions where spreads of the warp yarn 1 of the first portion on the second region side R21 and the first portion on the third region side R31 are maximized (see Wa1 to Wa3 in FIG. 2), in a predetermined area of the artificial blood vessel (for example, 1 mm × 1 mm), to calculate an average value thereof ((Wa1 + Wa2 + ... Wam) / m).

Moreover, in the artificial blood vessel, the weft yarn 2 is composed of a multifilament yarn, and in the second region R2 and the third region R3, the total number of filaments of the warp yarns 1 crossing over the plurality of weft yarns 2 (the warp yarn 1d, 1e, 1j, 1k in FIG. 1) may be 1.5 times or more, preferably 1.5 to 3.0 times the number of filaments per single weft yarn 2. Here, “the total number of filaments of the warp yarns 1 crossing over the plurality of weft yarns 2″ mean, in a case where the number of warp yarns 1 crossing over the plurality of weft yarns 2 is one in one second region R2 or one third region R3, the number of filaments for the one warp yarn, and in a case where the number of warp yarns 1 crossing over the plurality of weft yarns 2 is two or more (for example, two or three), the total number of filament yarns for the plurality of warp yarns 1 (the number of filaments constituting the one warp yarn 1 multiplied by 2 or 3 which is the number of warp yarns). When the total number of filaments of the warp yarns 1 crossing over the plurality of weft yarns 2 are larger than the number of filaments per single weft yarn 2, the multifilament yarn of the warp yarn 1 is easier to spread than the multifilament yarn of the weft yarn 2, and the blood leakage resistance can be further enhanced. That is, the warp yarn 1 having a larger total number of filaments than the weft yarn 2 is bound by the weft yarn 2 that is thinner (namely, has a smaller number of filaments) than the warp yarn 1, at both ends of the first portion on the second region side R21 and the first portion on the third region side R31, in the extending direction D1 of the warp yarn 1. As a result, with the warp yarn 1 being bound by the thin weft yarn 2, a strong pressure is applied to the warp yarn 1, and the warp yarn 1 becomes more easily spread in the extending direction D2 of the weft yarn 2. Furthermore, when the total number of filaments of the warp yarns 1 and the number of filaments per single weft yarn 2 are provided in the above-described ratios, the number of the weft yarns 2 becomes smaller than the number of filaments of the warp yarns 1, therefore, it becomes easy to close up the weft yarns 2 in the extending direction D1 of the warp yarn 1 in weaving an artificial blood vessel. Therefore, by closing up the weft yarns 2 in the extending direction D1 of the warp yarn 1, the gaps (porosities) formed at the intersection part between the warp yarn 1 and the weft yarn 2 can be reduced, and the blood leakage amount itself can be reduced. Therefore, the blood leakage resistance can be dramatically enhanced by the synergistic effect of the reduction of the blood leakage amount itself by facilitating the closing up of the weft yarn 2 and the absorbability of blood leaking with the three-dimensional structure of the warp yarn 1.

In the present embodiment, in each of the second region R2 and the third region R3, the number of warp yarns 1 crossing over the plurality of weft yarns 2 is configured to be one, and the number of filaments per single warp yarn 1 in the second region R2 and the third region R3 is configured to be 1.5 times or more, preferably 1.5 to 3 times the number of filaments per single weft yarn. Specifically, the number of filaments per single weft yarn 2 can be 4 to 500, and the number of filaments per single warp yarn 1 can be 8 to 1000. As a result, in the second region R2 and the third region R3, multifilament yarns constituting the warp yarns 1 crossing over the plurality of weft yarns 2 are bundled into one in each of the second region R2 and the third region R3, the number of which is larger than the number of filaments of the weft yarns 2. It should be noted that, in the second region R2 and the third region R3, configurations of the warp yarn 1 and the weft yarn 2 are not particularly limited to the above-mentioned configurations as long as the total number of filaments of the warp yarns 1 crossing over the plurality of weft yarns 2 is configured to be 1.5 times or more the number of filaments per single weft yarn 2. For example, in the second region R2 and the third region R3, the number of warp yarns 1 crossing over the plurality of warp yarns 2 may be two or more, and the number of filaments per single warp yarn 1 may be 0.8 to 1.2 times the number of filaments per single weft yarn 2 (preferably the same number of filaments). Also in this case, when the number of warp yarns 1 crossing over the plurality of weft yarns 2 is two or more, in the second region R2 and the third region R3, the total number of filaments of the warp yarns 1 crossing over the plurality of weft yarns 2 is larger than the number of filaments per single weft yarn 2. Therefore, the similar effect as the above-mentioned effect can be obtained.

In the present embodiment, as shown in FIG. 1, the second region R2 has at least one warp yarn 1 (warp yarns 1c, 1i) extending so as to cross over (only) one weft yarn 2 and at least one warp yarn 1 (warp yarns 1d, 1j) crossing over a plurality of weft yarns 2, and multifilament yarns constituting a plurality of warp yarns 1 in the second region R2 are bundled by weft yarns 2 crossing over a plurality of warp yarns 1 (warp yarns 1c, 1d, or warp yarns 1i, 1j) in the second region R2, at both ends of the first portion on the second region side R21 in the extending direction D1 of the warp yarn 1. For example, both ends of the warp yarn 1c and the warp yarn 1d crossing over the weft yarns 2g, 2h, and 2i are bundled by the weft yarns 2f and 2j. Furthermore, the third region R3 has at least one warp yarn 1 (warp yarns 1f, 1l) extending so as to cross over (only) one weft yarn 2 and at least one warp yarn 1 (warp yarns 1e, 1k) crossing over a plurality of weft yarns 2, and multifilament yarns constituting a plurality of warp yarns 1 in the third region R3 are bundled by weft yarns 2 crossing over a plurality of warp yarns 1 (warp yarns 1d, 1f, or warp yarns 1k, 1l) in the third region R3, at both ends of the first portion on the third region side R31 in the extending direction D1 of the warp yarn 1. For example, both ends of the warp yarn 1e and the warp yarn 1f crossing over the weft yarns 2d, 2e, and 2f are bundled by the weft yarn 2c and 2g. In the case above, in the second region R2 and the third region R3, unlike the first region R1 having a plain weave structure, a plurality of warp yarns 1 (two in FIG. 1) are bundled (tied up) together on one surface of the artificial blood vessel. As a result, the spread of the weft yarn 2 in the extending direction D2, in the central portions of the first portion on the second region side R21 and the first portion on the third region side R31, becomes large, and an amount of blood that can be absorbed by the three-dimensional structure of the warp yarn 1 can be increased. Therefore, the blood leakage resistance can be further improved.

Moreover, in the present embodiment, in the second region R2 and the third region R3, the number of filaments of the warp yarns 1 (e.g., 1d, 1e, 1j, 1k) crossing over the plurality of weft yarns 2 is larger than the number of filaments per single weft yarn 2 (e.g., 1.5 to 3 times), and the number of filaments of the warp yarns 1 (e.g., 1c, 1f, 1i, 1l) crossing over only one weft yarn 2 is also larger than the number of filaments per single weft yarn 2. (e.g., 1.5 to 3 times). These two warp yarns 1 each of which has filaments larger than the number of filaments per single weft yarn 2 are bundled with one weft yarn 2 having a smaller number of filaments. As a result, a reaction force applied from the warp yarn 1 to the one weft yarn 2 becomes larger than the reaction force applied in a case of bundling one warp yarn and in a case of bundling warp yarns having a smaller number of filaments per single warp yarn. Therefore, for example, in a case where the artificial blood vessel is cut along the extending direction D2 of the weft yarn, the weft yarn 2 becomes less likely to fray from the cut portion when the artificial blood vessel is touched by a doctor or the like. Furthermore, as mentioned above, in the second region R2 and the third region R3, the warp yarn 1 spreads in the extending direction D2 of the weft yarn 2 to cover the surface of the weft yarn 2. As a result, the weft yarn 2 becomes less likely to be exposed on the surface of the artificial blood vessel, and when a doctor or the like touches the artificial blood vessel, chances of touching the weft yarn 2 are reduced, so that the weft yarn 2 is suppressed from fraying from the cut portion of the artificial blood vessel.

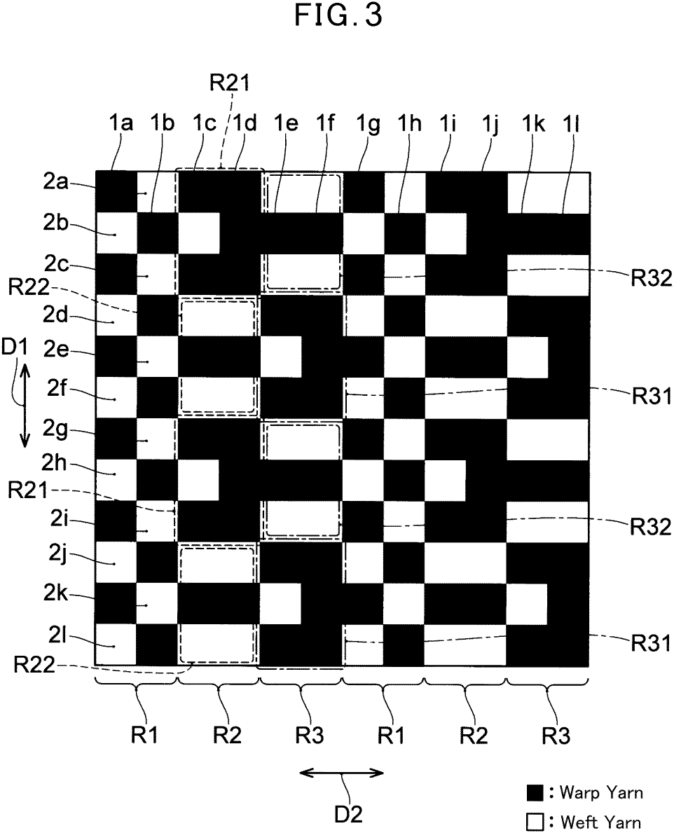

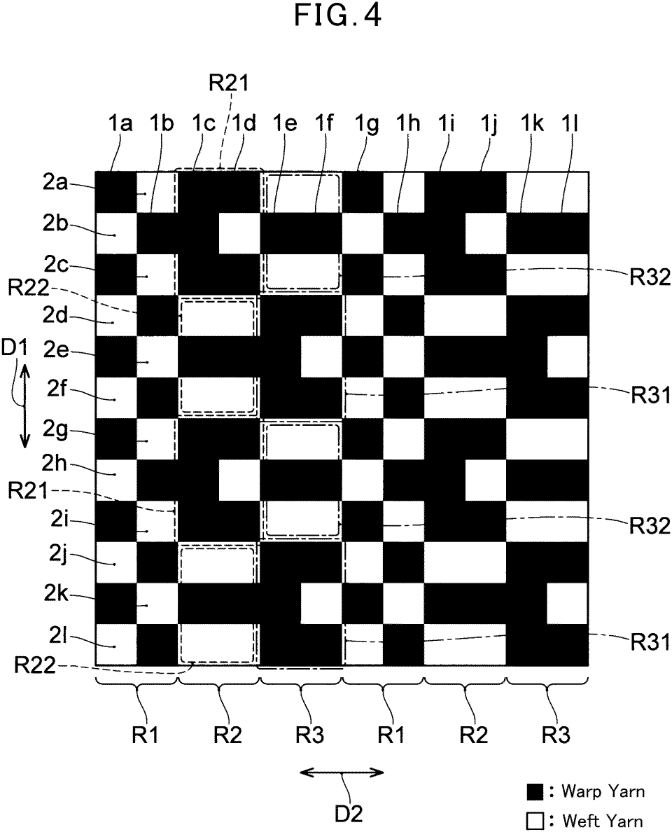

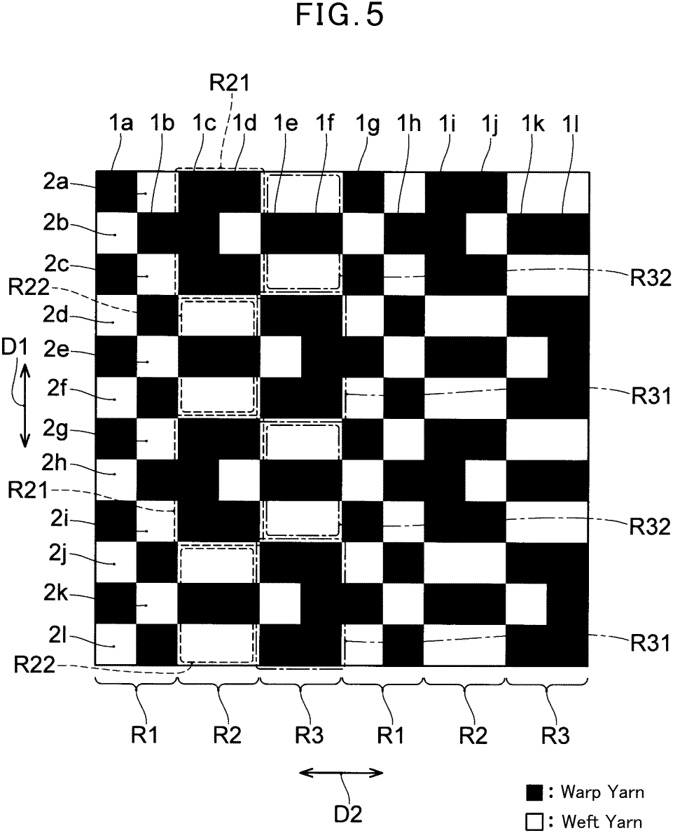

It should be noted that the second region R2 and the third region R3 are not limited to the arrangements shown in FIG. 2 and may be configured as in the modified examples shown in FIGS. 3 to 5. FIG. 3 shows that the warp yarn 1e (warp yarn 1k) and the warp yarn 1f (warp yarn 1l) in the first portion on the third region side R31 in FIG. 2 are reversed (left and right reversed) in the extending direction D2 of the weft yarn 2. FIG. 4 shows that the warp yarn 1c (warp yarn 1i) and the warp yarn 1d (warp yarn 1j) in the first portion on the second region side R21 in FIG. 2 are reversed (left and right reversed) in the extending direction D2 of the weft yarn 2. FIG. 5 shows that the warp yarn 1c (warp yarn 1i) and the warp yarn 1d (warp yarn 1j) in the first portion on the second region side R21 in FIG. 2 are reversed (left and right reversed) in the extending direction D2 of the weft yarn 2, and the warp yarn 1e (warp yarn 1k) and the warp yarn 1f (warp yarn 1l) in the first portion on the third region side R31 are reversed (left and right reversed) in the extending direction D2 of the weft yarn 2. As described above, the artificial blood vessel is not limited to the illustrated structures as long as it satisfies the features of claims and has the technical idea of the present invention, and it may have structures other than the illustrated structures.

REFERENCE SIGNS LIST

| | |
|---|---|
| 1, 1a, 1b, 1c, 1d, 1e, 1f, 1g, 1h, 1i, 1j, 1k, 1l | Warp yarn |
| 2, 2a, 2b, 2c, 2d, 2e, 2f, 2g, 2h, 2i, 2j, 2k, 2l | Weft yarn |
| D1 | Extending direction of warp yarn |
| D2 | Extending direction of weft yarn |
| R1 | First region |
| R2 | Second region |
| R21 | First portion on second region side |
| R22 | Second portion on second region side |
| R3 | Third region |
| R31 | First portion on third region side |
| R32 | Second portion on third region side |

1. An artificial blood vessel having a warp yarn and a weft yarn,
the artificial blood vessel alternatingly having, in an extending direction of the weft yarn:
a first region in which the warp yarn and the weft yarn are woven in a plain weave,
a second region having a first portion of the second region, wherein, the warp yarn crosses over a plurality of weft yarns and a second portion of the second region wherein the warp yarn extends so as to cross over one weft yarn, and
a third region having a first portion of the third region wherein the warp yarn crosses over a plurality of weft yarns and a second portion of the third region wherein the warp yarn extends so as to cross over one weft yarn,
wherein the first portion of the second region is adjacent to the second portion of the third region in the extending direction of the weft yarn, and the second portion of the second region is adjacent to the first portion the third region in the extending direction of the weft yarn, and
wherein the warp yarn is composed of a multifilament yarn.

2. The artificial blood vessel of claim 1, wherein the first portion of the second region and the first portion of the third region are configured to extend continuously in an extending direction of the warp yarn in a zigzag shape.

3. The artificial blood vessel of claim 1, wherein the second region has at least one warp yarn extending so as to cross over one weft yarn and at least one warp yarn crossing over a plurality of weft yarns,
wherein multifilament yarns constituting a plurality of warp yarns in the second region are bundled by weft yarns crossing over a plurality of warp yarns in the second region, at both ends of the first portion of the second region in the extending direction of the warp yarn,
wherein the third region has at least one warp yarn extending so as to cross over one weft yarn and at least one warp yarn crossing over a plurality of weft yarns, and
wherein multifilament yarns constituting a plurality of warp yarns in the third region are bundled by weft yarns crossing over a plurality of warp yarns in the third region, at both ends of the first portion of the third region in the extending direction of the warp yarn.

4. The artificial blood vessel of claim 1, wherein the weft yarn is composed of a multifilament yarn, and
wherein a total number of filaments of warp yarns crossing over a plurality of weft yarns is 1.5 times or more the number of filaments per single weft yarn, in the second region and the third region.

5. The artificial blood vessel of claim 4, wherein the number of warp yarns crossing over a plurality of weft yarns is one, the number of filaments per single weft yarn is 4 to 500, and the number of filaments per single warp yarn is 8 to 1000, in the second region and the third region.

6. The artificial blood vessel of claim 4, wherein the number of warp yarns crossing over a plurality of weft yarns is two or more, and the number of filaments per single warp yarn is 0.8 to 1.2 times the number of filaments per single weft yarn, in the second region and the third region.

7. The artificial blood vessel of claim 1, wherein an average width of the maximum spread of the warp yarn of the first portion of the second region and the first portion of the third region in the extending direction of the weft yarn is larger than an average width of the maximum spread of the warp yarn in the first region in the extending direction of the weft yarn.
